(19) United States
(12) Patent Application Publication
(10) Pub. No.: US 2010/0196280 A1
(43) Pub. Date: Aug. 5, 2010

(54) FUNCTIONALIZED SOLID POLYMER NANOPARTICLES FOR DIAGNOSTIC AND THERAPEUTIC APPLICATIONS

(76) Inventors: Katrin Claudia Fischer, Berlin (DE); Sascha General, Berlin (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 12/303,805

(22) PCT Filed: Jun. 7, 2007
(86) PCT No.: PCT/EP07/05258

§ 371 Date: Mar. 25, 2010

Related U.S. Application Data
(60) Provisional application No. 60/811,756, filed on Jun. 8, 2006.

(51) Int. Cl. A61K 49/06; A61K 9/14; A61K 31/661; A61K 49/12; A61P 35/00; A61P 29/00; A61K 31/675; A61K 31/7068; A61K 31/475; A61K 31/7048; A61K 33/24; A61K 31/282; A61K 38/09; A61K 31/616; A61K 31/573; A61K 31/63; A61K 39/44; A61K 49/04; A61K 49/22; B29C 59/10

(52) U.S. Cl. 424/9.3; 424/501; 514/110; 424/9.6; 514/83; 514/49; 514/283; 514/27; 424/649; 514/492; 514/15; 514/165; 514/179; 514/155; 424/178.1; 424/9.4; 424/9.5; 204/164; 977/773; 977/915; 977/904

(57) ABSTRACT

The present invention describes polymer nanoparticles with a cationic surface potential, in which both hydrophobic and hydrophilic pharmaceutically active substances can be encapsulated. The hydrophilic and thus water-soluble substances are encapsulated in the particle core by co-precipitation through ionic complexing with a charged polymer. Both therapeutic agents and diagnostic agents can be used as pharmaceutically active substances for encapsulation. The cationic particle surface permits stable, electrostatic surface modification with partially oppositely charged compounds, which can contain target-specific ligands for improving passive and active targeting.

The present invention describes polymer nanoparticles with cationic surface potential, in which both hydrophobic and hydrophilic pharmaceutically active substances can be encapsulated. By ionic complexing with a charged polymer, the hydrophilic and thus water-soluble substances are enclosed in the particle core by co-precipitation. Both therapeutic agents and diagnostic agents can be used as pharmaceutically active substances for encapsulation. The cationic particle surface permits stable, electrostatic surface modification with partially oppositely charged compounds, which can contain target-specific ligands to improve passive and active targeting.

### BACKGROUND OF THE INVENTION

The special properties of nanoparticle drug delivery systems are based primarily on their small size, so that various physiological barriers can be overcome [Fahmy T. M., Fong P. M. et al., Mater. Today, 2005; 8(8): 18-26]. The associated altered distribution in the organism can be used to advantage e.g. both for diagnosis and for therapy of various neoplastic diseases. Nanoparticle systems that can be used both for detecting and for treating diseases are termed theranostics (=therapeutic agents+diagnostic agents). The associated therapeutic monitoring will in future permit faster recognition of resistance to therapy and greatly improve patient recovery through early use of alternative therapies [Emerich D. F., Thanos C. G., Curr. Nanosci., 2005; 1: 177-188].

The cytostatics represent a substance class that is used very successfully in tumor therapy. All of the body's rapidly dividing cells, including tumor cells, are damaged by these substances. However, this not only leads to death of the tumor cells, it also often affects other vital organs and tissues such as the bone marrow, mucosae or cardiac vessels. The associated undesirable toxicity is often the dose-limiting factor in the therapy [Silacci D., Neri M., Modern Biopharmaceuticals: Design, development and optimization, Volume 3, Part V, Wiley-VCH, Weinheim, 2005; 1271-1299].

It has been shown that by encapsulating such substances in nanoparticle systems there is less damage to healthy tissues and a locally higher concentration of the active substance in the tumor tissue can be achieved [Silacci D., Neri M., Modern Biopharmaceuticals: Design, development and optimization, Volume 3, Part V, Wiley-VCH, Weinheim, 2005; 1271-1299]. The successful introduction of the marketed product Doxil® is proof of the clinical advantage of this nanoformulation.

The enhanced permeation and retention effect (EPR-effect) has mainly been considered to be responsible for this. This EPR-effect had already been described in 1986 by Matsumura and Maeda as a strategy for targeted drug accumulation in solid tumors [Matsumura Y., Maeda H., Cancer Res., 1986; 46: 6387-6392][Maeda H., Adv. Enzyme Regul., 2001; 41: 189-207]. This involves a passive accumulation mechanism, which utilizes the structural peculiarities of tumoral tissue or also inflamed tissue [Ulbrich K., Subr V., Adv. Drug Deliv. Rev., 2004; 56(7): 1023-1050].

In particular, owing to its rapid growth and various messenger substances, tumoral tissue is generally characterized by a fenestrated “holey” tissue structure and absence of lymphatic drainage. Depending on the type of tumor, the size of the fenestrations is generally put at between 380 nm and 780 nm, so this range is also termed nanosize window [Hobbs S. K., Monsky W. L. et al., Proc. Natl. Acad. Sci. USA, 1998; 95: 4607-4612][Brigger I., Dubernet C. et al., Adv. Drug Deliv. Rev., 2002; 54(5): 631-651]. In contrast, normal tissues such as heart, brain or lung possess so-called tight junctions, which, having a diameter of less than 10 nm (generally 2 nm to 4 nm), are impermeable to colloidal drug vehicles [Hughes G. A., Nanomedicine, 2005; 1(1): 22-30].

Nanoparticles circulating in the bloodstream are thus able to accumulate passively in tumoral tissue by diffusion from the bloodstream. Absence of lymphatic drainage promotes long-lasting accumulation in the tumor or prevents rapid washout of the nanoparticles (EPR-effect).

For this accumulation mechanism to be possible, the nanoparticles must circulate in the bloodstream for a sufficient length of time. This requires particle sizes between approx. 10 nm and 380 nm and suitable particle surfaces. For example, pegylated particle surfaces can prevent the body's own proteins identifying the particles as foreign, with rapid elimination via the organs of the reticulo-endothelial system (RES) [Otsuka H. et al., Adv. Drug Deliv. Rev., 2003; 55(3): 403-419]. By using active ligands on the particle surface (e.g. antibodies), tissue-specific accumulation can be further optimized [Nobs L. et al., Pharm. Sci., 2004; 93: 1980-1992] [Yokoyama M., J. Artif. Organs, 2005; 8: 77-84].

For the active substances to be absorbed into the cell, yet another physiological barrier, the cell membrane, must be overcome. One of the difficulties for many medicinal substances is that the cell possesses very effective transport mechanisms (e.g. P-glycoprotein) for ejecting foreign or toxic substances. If, however, with the aid of nanoparticles, the active substance is brought into the cell by endocytosis, ejecting transporters can be avoided and so-called multidrug resistance (MDR) can be prevented [Bharadwaj V., J. Biomed. Nanotechnol., 2005; 1: 235-258] [Huwyler J. et al., J. Drug Target., 2002; 10(1): 73-79].

Nanoparticles are generally incorporated in the cell by endocytosis. For this reason, after the absorption process the particles are contained in endosomes or endolysosomes [Koo O. M. et al., Nanomedicine, 2005; 1(3):193-212]. Provided no release of the particles from the endolysosomes occurs, there is enzymatic degradation of active substance and colloidal vehicle system within the vesicles. Endolysosomal release of the particles and hence of the active substance is therefore essential for the intracellular therapeutic effect.

The release properties of the active substance from the nanoparticle can additionally be controlled by appropriate selection of the polymer. A nanoparticle formulation can thus minimize the frequency of application and lead to a reduction of the therapeutically necessary dose. Furthermore, undesirable peak plasma levels can be avoided by encapsulation in nanoparticles, and delayed release can be achieved.

To summarize, the following advantages are decisive for the development of polymer nanoparticles: (i) targeted accumulation of the active substances (a) passively by the EPR-effect, (b) actively by means of tissue- or cell-specific ligands, e.g. antibodies, (ii) controllable active substance release by appropriate selection of the polymer, (iii) avoidance of large fluctuations in plasma levels, (iv) lowering the dose or increasing the effectiveness at equal dose, (v) fewer side effects and improved safety profile, (vi) reduced frequency of application with improved compliance and (vii) circumventing resistance mechanisms (P-glycoprotein) [Rosen H., Abribat T., Nature Reviews Drug Discovery, 2005 May; 4(5): 381-5][McLennan D. N., Porter C. J. H. et al., Drug Discovery Today: Technologies, 2005 Spring; 2(1): 89-96]. A nanoparticle system, which already fulfils all the advantages described, has not yet been developed in the state of the art. Moreover, it is clear from the great variety of nanoparticle vehicle systems described in the literature that at the present time there is no optimum nanoformulation for all problems that may be envisaged. In addition to size, the overall structure of the particles, the matrix-forming substances and especially their surface are of decisive importance for the behavior in vivo [Choi S. W., Kim W. S., Kim J. H., Journal of Dispersion Science and Technology, 2003; 24(3&4): 475-487]. Furthermore, the physicochemical properties of different active substances vary considerably. Accordingly, there is still a need for the development of colloidal drug vehicle systems with improved properties.

For future therapeutic approaches it will be necessary to prove, for example by diagnostic detection of the distribution of the particles in the organism, that accumulation mainly occurs in the diseased tissue (e.g. in the tumor). Imaging techniques such as sonography, X-ray diagnosis, sectional-imaging techniques (CT, MRT) and nuclear medicine (PET, SPECT) are available for detection in vivo. Another, relatively new method is optical imaging, the detection principle of which is based on the use of near-infrared fluorescence. It is a non-invasive method, which operates without ionizing radiation, and in comparison with methods such as MRT is very cost-effective and is less time-consuming. The NIR dyes developed for such applications, such as Indocyanine Green, have very good solubility in water, so it is difficult for them to be encapsulated efficiently in a hydrophobic polymer matrix. The reason for this is the rapid change of the hydrophilic substance to the aqueous phase, for example during production by nanoprecipitation.

For the encapsulation of hydrophilic substances in nanoparticles, only a few technologies are available, and they have various shortcomings. The amphiphilic character of liposomes or polymerosomes makes it possible, for example, to enclose hydrophilic substances in the aqueous interior of the particles, whereas hydrophobic compounds can be incorporated in the membrane. Owing to localization in the core or in the shell of the particles, loading is very limited and therefore is generally inadequate. Another disadvantage is that, in particular, hydrophilic substances in an aqueous environment are quickly washed out of such systems.

Alternative encapsulation of water-soluble substances in polyelectrolyte complexes is only possible to a limited extent, because dyes such as Indocyanine Green (ICG) are small molecules with few charged groups, so that insufficient charges are available for electrostatic complexing. Furthermore, polyelectrolyte complexes in aqueous solution are very dynamic systems, so they generally have inadequate colloidal stability in plasma [Thünemann A. F. et al., Adv. Polym. Sci., 2004; 166: 113-171].

As already described, in future it will be necessary to demonstrate, by means of a diagnostic nanoparticle, that accumulation of the particles occurs mainly at the target location, for example the tumor. If this proof is provided, a therapeutically active substance can be encapsulated in one and the same system and can achieve a maximum therapeutic effect at the site of action, since the desired distribution of the nanoparticles had already been demonstrated using the diagnostic system. To avoid altering the distribution properties of the particles it is therefore important to be able to use one and the same nanoparticle system for the diagnostic detection and the subsequent treatment. As already described, there are several different nanoparticle systems, which are however suitable either for the encapsulation of hydrophilic or hydrophobic substances. It is known from the literature that just slight changes in properties of the nanoparticles, such as particle size, surface material, type of matrix polymer or even the use of a different surfactant have an enormous influence on the distribution of the particles in the body. Therefore it is important to be able to carry out diagnosis, therapy and perhaps even monitoring of the treatment with one and the same system.

Ideally, therefore, it should be possible to encapsulate both water-soluble dyes for diagnosis and therapeutic substances, which owing to their hydrophobic properties generally have low solubility in water, effectively and with sufficient stability against washing-out, in one and the same nanoparticle system

An additional technological challenge is to ensure, by the use of suitable surfaces, on the one hand sufficient particle stability and on the other hand specific accumulation in the target tissue. Whether it remains at the site of accumulation (target tissue) depends on, among other things, how well the particles are absorbed into the tissue and the cell.

It is known from the literature that cationic particle surfaces promote uptake into the cell [Mounkes L. C. et al., J. Biol. Chem., 1998; 273(40): 26164-26170] [Mislick K. A., Baldeschwieler J. D., Proc. Natl. Acad. Sci. USA, 1996; 93: 12349-12354]. This is because of electrostatic interactions between the negatively charged cell membrane (sulfated proteoglycans) and the cationic particle surface (generally protonated amine functions). In addition, polymers or substances bearing amino groups are known to possess endoosmolytic activity, i.e. they promote intracellular release of the particles from the endolysosomes by damaging the endolysosome membrane [DeDuve C. et al., Biochem. Pharmacol., 1974; 23:2495-2531]. If the particles remain within the cell in the endolysosomes, the particle matrix and the substances incorporated therein are degraded by the cell's own enzymes. Endolysosomal release of the encapsulated active substances is therefore essential for the therapeutic effect.

There is the problem, however, that sometimes severe toxicological effects have been described during in vivo studies of polyplexes and lipids with strongly cationic charged surfaces [Kircheis S. et al., J. Gene Med., 1999; 1: 111-120][Ogris M. et al., Gene Ther., 1999; 6: 595-605]. The reason is that cationic particles aggregate with negatively charged erythrocytes and this leads to blockage of the blood vessels. In addition, this generally leads to considerable accumulation in the lung, through which the particles pass as the first capillary bed after i.v. application [Kircheis R. et al., Drug Deliv. Rev., 2001; 53(3): 341-58]. In this case there is a risk of pulmonary embolism, promoted by agglomerates of particles and erythrocytes or other blood components [Kircheis S. et al., J. Gene Med., 1999; 1: 111-120][Ogris M. et al., Gene Ther., 1999; 6: 595-605].

Ideally, nanoparticle systems should therefore be produced with cationic surface properties, without possible toxicologically questionable properties hampering in-vivo use. In addition, the particle surface must be inconspicuous to the body's own defense mechanisms (opsonins, RES), for the first time permitting a sufficiently long circulation time, which is a prerequisite for corresponding accumulation of the particles from the bloodstream in the target tissue. The nanoparticle systems should also promote uptake into the target cell and endolysosomal release.

A further difficulty in the production of nanoparticle systems is to apply suitable substances or target structures on the particle surface.

Often the surface of the particles is modified by means of covalent coupling reactions. A prerequisite for this is the presence of functional groups on the polymer backbone or on the particle surface, which can be joined irreversibly to the target molecule by chemical coupling reactions [Nobs L. et al., J. Pharm. Sci., 2004; 93: 1980-1992]. As the stability of colloidal dispersions is often greatly reduced by the reagents or under the reaction conditions, the chemical processes are generally costly and problematic [Koo O. M. et al., Nanomedicine, 2005; 1(3):193-212][Choi S. W. et al., J. Dispersion Sci. Technol., 2003; 24(3&4): 475-487]. The covalent coupling of molecules and particle surfaces must additionally be specially suited for each new molecule to be applied to the surface, in order to avoid possible unwanted chemical reactions. Avoidance of organic solvents, which are often used for covalent coupling reactions, is also desirable for reducing environmental pollution and for simplifying execution of the reaction.

Ideally, therefore, surface modification should be non-covalent, simple to carry out, and thus flexible but nevertheless stable.

The colloidal systems known from the literature are generally only suitable for the encapsulation of hydrophobic substances or alternatively hydrophilic substances. In the case of the frequently used covalent surface modification of the particles, there is little flexibility regarding use of very varied surface structures on one and the same core particle. In addition, the ligands for specific accumulation often adversely affect uptake in the actual tumor tissue and in particular on cellular uptake. Although the particles ensure adequate circulation and are accumulated well, passively or actively, in the target tissue, generally internalization and endolysosomal release are not optimal [van Osdol W., Cancer Res., 1991; 51: 4776-4784] [Weinstein J. N. et al., Cancer Res., 1992; 52(9): 2747-2751].

Accordingly, there is still a need for pharmaceutical, nanoparticle formulations, which: (i) encapsulate both water-soluble and sparingly water-soluble pharmaceutically active substances, effectively and with sufficient stability against washout, (ii) permit surface modification that is non-covalent, simple to carry out (flexible) and nevertheless stable, (iii) permit a sufficient circulation time (iv), are absorbed effectively into the target tissue and (v) are released intracellularly there, from the endolysosomes.

One task of the invention was therefore to make available an improved pharmaceutical formulation in which, on the one hand, both hydrophilic and hydrophobic active substances can be encapsulated. On the other hand, flexible and sufficiently stable surface modification should permit optimum accumulation in the diseased tissue. In order to be able to achieve a maximum diagnostic or therapeutic effect, such a colloidal system must also be taken up efficiently into the target tissue and into the individual cells, where endolysosomal release can take place. Furthermore, the methods of production should be practicable, to permit production in a reasonable time and at acceptable cost.

### DESCRIPTION OF THE INVENTION

The invention relates to polymer nanoparticles with a cationic surface potential, containing a cationic polymer and a polymer that is sparingly water-soluble, characterized in that said polymer nanoparticles contain diagnostic and/or to therapeutic agents.

It was found, surprisingly, that by co-precipitation of a water-soluble cationic polymer with a sparingly water-soluble polymer, stable polymer nanoparticles can be produced, which have a cationically functionalized surface. Moreover, it was surprising, in the sense of the invention, that both hydrophilic, readily water-soluble substances and hydrophobic, sparingly water-soluble substances could be encapsulated in the polymer matrix of the nanoparticles described above. Unexpectedly, ionic complexing of water-soluble substances of low molecular weight with the charged cationic polymer led to successful encapsulation in the polymer matrix of the particles by nanoprecipitation. In the sense of the invention, substances that are suitable for the diagnosis and/or therapy of various diseases can be encapsulated in the polymer particles. Furthermore, it was found that the cationically functionalized particle surface can be electrostatically surface-modified stably and flexibly with a partially oppositely charged compound. The invention described is therefore suitable for the recognition of diseases (diagnosis), for the treatment of diseases (therapy), as well as for monitoring the treatment. In addition, the invention comprises a suitable pharmaceutical form, using pharmaceutically acceptable excipients that are required for the particular pharmaceutical form. The pharmaceutical form developed in the sense of the invention can be used in humans or animals via various routes of administration. The necessary application systems also form part of the invention described here.

The composition of the nanoparticles comprises a sparingly water-soluble polymer, which is preferably a biodegradable polymer or a mixture of various biodegradable polymers. The biodegradable polymer can be described in terms of individual monomer units, which form said polymer by polymerization or other processes. Furthermore, the polymer can be defined by its name.

In one embodiment, the sparingly water-soluble polymer is derived from the group of the natural and/or synthetic polymers or from homo- and copolymers of corresponding monomers. In particular, the polymer is derived from the alkylcyanoacrylate group, for example the butylcyanoacrylates and the isobutylcyanoacrylates, the acrylates, such as the methacrylates, the lactides, for example the L-lactides or DL-lactides, the glycolides, the caprolactones such as the ε-caprolactones and others.

In another embodiment, said polymer or part of the polymer is selected from the group comprising polycyanoacrylates and polyalkylcyanoacrylates (PACA), for example polybutylcyanoacrylate (PBCA), polyesters, for example poly(DL-lactides), poly(L-lactides), polyglycolides, polydioxanones, polyoxazolines, poly(glycolides-co-trimethylene-carbonates), polylactide-co-glycolides (PLGA), for example poly(L-lactides-co-glycolides) or poly(DL-lactides-co-glycolides), poly(L-lactides-co-DL-lactides), poly(glycolides-co-trimethylene), poly(carbonates-co-dioxanones), alginic acid, hyaluronic acid, polysialic acid, acid cellulose derivatives, acid starch derivatives, polysaccharides for example dextrans, alginates, cyclodextrins, hyaluronic acid, chitosans, acid polyamino acids, polymeric proteins, for example collagen, gelatin or albumin, polyamides for example poly(aspartic acid), poly(glutamic acid), polylysines, poly(iminocarbonates) (poly(carbonates) derived from tyrosine, poly(β-hydroxybutyrate), polyanhydrides, for example polysebacic acid (Poly(SA)), poly(adipic acid), poly(CPP-SA), poly(CPH), poly(CPM), aromatic polyanhydrides, polyorthoesters, polycaprolactones for example poly-ε- or γ-caprolactones, polyphosphoric acid such as polyphosphates, polyphosphonates, polyphosphazenes, poly(amide-enamines), azopolymers, polyurethanes, polyorthoesters, dendrimers, pseudopolyamino acids as well as all mixtures and copolymers of said compounds.

In a preferred embodiment, the sparingly water-soluble polymer is from the group of the polyalkylcyanoacrylates (PACA).

The constitution of these polyalkylcyanoacrylates is shown by the structure given below (Formula 1), where the stated residue R preferably denotes linear and branched alkyl groups with 1 to 16 carbon atoms, a cyclohexyl, benzyl or phenyl group.

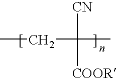

Formula 1: structural formula of PACA, n=5-20000, preferably n=5-6000, or n=5-100

In another preferred embodiment, the sparingly water-soluble polymer is a polybutylcyanoacrylate (PBCA) (Formula 2).

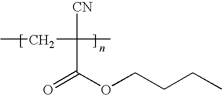

Formula 2: structural formula of PBCA; n=5-20000 preferably n=5-6000, or n=5-100

In the sense of the invention, the sparingly water-soluble polymer forms the greater part of the polymer matrix of the particles.

Surprisingly, it was found that by incorporating compounds with amino groups, especially a cationic polymer, in a sparingly water-soluble, solid polymer matrix, nanoparticles with a cationically charged surface potential (zeta potential) are produced.

In one embodiment, the cationic polymer is derived from the group of the natural and/or synthetic polymers or from homo- and copolymers of corresponding monomers.

Polymers with free primary, secondary or tertiary amino groups, which can form salts with any low-molecular acids, the salts being soluble in aqueous-organic solvents, are suitable as cationic polymers in the sense of this invention. Polymers or salts thereof that carry quaternary ammonium groups and are soluble in organic solvents, are also suitable.

In a preferred embodiment, the following groups of cationic polymers, polycations and polyamine compounds or polymers from homo- and copolymers of corresponding monomers are particularly suitable: modified natural cationic polymers, cationic proteins, synthetic cationic polymers, aminoalkanes of varying chain length, modified cationic dextrans, cationic polysaccharides, cationic starch or cellulose derivatives, chitosans, guar derivatives, cationic cyanoacrylates, methacrylates and methacrylamides and monomers and comonomers such as can be used for forming corresponding suitable compounds and the corresponding salts, which can be formed with suitable inorganic or low-molecular organic acids.

This includes in particular: diethylaminoethyl-modified dextrans, hydroxymethylcellulosetrimethylamine, polylysine, protamine sulfate, hydroxyethylcellulosetrimethylamine, polyallylamines, protamine chloride, polyallylamine hydrated salts, polyamines, polyvinylbenzyltrimethylammonium salts, polydiallyldimethylammonium salts, polyimidazoline, polyvinylamine and polyvinylpyridine, polyethyleneimine (PEI), putrescine (butane-1,4-diamine), spermidine (N-(3-aminopropyl)butane-1,4-diamine), spermine (N,N′-bis(3-aminopropyl)butane-1,4-diamine) dimethylaminoethylacrylate, poly-N,N-dimethylaminoethylmethacrylate, dimethylaminopropylacrylamide, dimethylaminopropylmethacrylamide, dimethylaminostyrene, vinylpyridine and methyldiallylamine, poly-DADMAC, guar, deacetylated chitin and the corresponding salts that can be formed with suitable inorganic or low-molecular organic acids. Suitable acids for salt formation are e.g.: hydrochloric acid, sulfuric acid, but in particular also acetic acid, glycolic acid or lactic acid.

In one embodiment, the compound bearing amino groups, in particular a cationic polymer, can be dissolved in an organic solvent that is completely miscible with water, preferably acetone, methanol, ethanol, propanol, dimethylsulfoxide (DMSO), or in a mixture of these solvents with water.

In an especially preferred embodiment, the polymer nanoparticles contain, as compound bearing amino groups, a cationically modified polyacrylate (poly-N,N-dimethylaminoethylmethacrylate, P(DMAEMA)) (Formula 3).

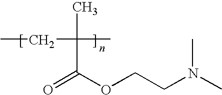

Formula 3: Structural formula of P(DMAEMA), n=5-20000, preferably n=5-6000, or n=5-100

The biologically degradable, cationically modified polyacrylate P(DMAEMA) is encapsulated in the polymer matrix, in particular the PBCA-polymer matrix, by nanoprecipitation. The surface of the resultant nanoparticles has, owing to the amino groups of the cationic polymer, a positive (cationic) surface potential (zeta potential). The cationic particle surface ensures good cellular uptake and permits flexible electrostatic surface modification with partially anionically charged compounds.

In another preferred embodiment, the polymer nanoparticles contain, as cationic polymer, a modified polyacrylate poly(dimethylaminopropyl methacrylamide).

In another preferred embodiment, the polymer nanoparticles contain, as cationic polymer, polyethyleneimine (PEI) of varying molecular weights, in particular 1.8 kDa, 10 kDa, 70 kDa and 750 kDa (Formula 4).

PEI is a polycation that is frequently used in the area of non-viral gene therapy for DNA-polyplexes (PEK) and accordingly has been investigated a great deal [Remy J. -S. et al., Adv. Drug Deliv. Rev., 1998; 30(1-3): 85-95].

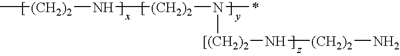

Formula 4: General structural formula for branched polyethyleneimine, where x, y and z=10-50%, preferably x, y and z=20-40% with the total coming to 100%.

Owing to the encapsulation of the cationic polyelectrolyte PEI in the PBCA-polymer matrix, the particle shell comprises PEI polymer chains, which produce a cationic surface potential.

Additionally, according to the invention, along with the aforementioned cationic polymers or compounds with amino groups, it is also possible for diagnostic or therapeutic substances to be encapsulated in the polymer matrix by nanoprecipitation.

As diagnostic substances for encapsulation, the following classes of substances can be employed for various molecular imaging methods, and in particular we may mention contrast agents or tracers for the following methods for molecular imaging: optical imaging, e.g. DOT (diffuse optical imaging), US (ultrasound imaging), OPT (optical projection tomography), near-infrared fluorescence imaging, fluorescence protein imaging and BLI (bioluminescence imaging) and magnetic resonance tomography (MRT, MRI) or X-raying. However, other methods are also conceivable. Encapsulation of a suitable diagnostic substance from the stated groups of substances permits detection of the particles in vitro and/or in vivo.

In a preferred embodiment, the diagnostic agent comprises dyes, in particular selected from the following group: fluorescein, fluorescein isothiocyanate, carboxyfluorescein or calcein, tetrabromofluoresceins or eosins, tertaiodofluorescein or erythrosine, difluorofluorescein, such as Oregon Green™ 488, Oregon Green™ 500 or Oregon Green™ 514, carboxyrhodol (Rhodol Green™) dyes (U.S. Pat. No. 5,227,487; U.S. Pat. No. 5,442,045), carboxyrhodamine dyes (Rhodamine Green™ dyes) (U.S. Pat. No. 5,366,860), 4,4-difluoro-4-bora-3a,4a-diaza-indacenes, e.g. Dodipy FL, Bodipy 493/503 or Bodipy 530/550 and derivatives thereof (U.S. Pat. No. 4,774,339; U.S. Pat. No. 5,187,288; U.S. Pat. No. 5,248,782; U.S. Pat. No. 5,433,896; U.S. Pat. No. 5,451,663), polymethine dyes, coumarin dyes, e.g. Coumarin 6, 7-amino-4-methylcoumarin, metal complexes of DTPA or tetraazamacrocyclene (Cyclene, Pyvlene) with terbium or europium or tetrapyrrole dyes, in particular porphyrins.

In an especially preferred embodiment, the diagnostic substance comprises a fluorescence-active dye.

In a quite especially preferred embodiment, the diagnostic agent comprises a fluorescent near-infrared (NIR) dye. These NIR dyes, which are preferably used for optical imaging, absorb and emit light in the NIR region between 650 nm and 1000 nm. The preferred dyes belong to the class of the polymethine dyes and are selected from the following groups: carbocyanines for example diethyloxacarbocyanine (DOC), diethyloxadicarbocyanine (DODC), diethyloxatricarbocyanine (DOTC), indo-di- or indotricarbocyanines, tricarbocyanines, merocyanines, oxonol dyes (WO 96/17628), rhodamine dyes, phenoxazine or phenothiazine dyes, tetrapyrrole dyes, in particular benzoporphyrins, chorines and phthalocyanines.

The stated dyes, preferred in this invention, can either be used as acids or as salts. Suitable inorganic cations or counterions for these dyes are for example the lithium ion, the potassium ion, the hydrogen ion and in particular the sodium ion. Suitable cations of organic bases are, among others, those of primary, secondary or tertiary amines, for example ethanolamine, diethanolamine, morpholine, glucamine, N,N-dimethylglucamine and in particular N-methylglucamine and polyethyleneimine. Suitable cations of amino acids are for example those of lysine, of arginine and of ornithine and the amides of otherwise acid or neutral amino acids.

Also, the preferred dyes can be used as their bases or salts.

In a quite especially preferred embodiment, the diagnostic substance comprises a carbocyanine dye. The general structure of the carbocyanines is described as follows (Formula 8).

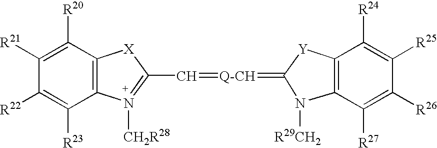

where Q is a fragment

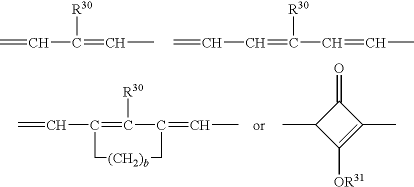

where R³⁰ stands for a hydrogen atom, a hydroxyl group, a carboxyl group, an alkoxy residue with 1 to 4 carbon atoms or a chlorine atom, R³¹ stands for a hydrogen atom or an alkyl residue with 1 to 4 carbon atoms,
X and Y, independently of one another, stand for a fragment —O—, —S—, —CH═CH— or —C(CH₂R³²) (CH₂R³³)⁻,
R²⁰ to R²⁹, R³² and R³³, independently of one another, stand for a hydrogen atom, a hydroxyl group, a carboxyl residue, a sulfonic acid residue or a carboxyalkyl, alkoxycarbonyl or alkoxyoxoalkyl residue with up to 10 carbon atoms or a sulfoalkyl residue with up to 4 carbon atoms, or for a non-specifically binding macromolecule, or for a residue of general formula VI —(O)v-(CH₂)o-CO—NR³⁴—(CH₂)s-(NH—CO)q-R³⁵ (VI), provided that with X and Y both denoting O, S, —CH═CH— or —C(CH₃)₂— at least one of the residues R²⁰ to R²⁹ corresponds to a non-specifically binding macromolecule or to general formula VI where
o and s are equal to 0 or, independently of one another, stand for an integer from 1 to 6,
q and v, independently of one another, stand for 0 or 1,
R³⁴ represents a hydrogen atom or a methyl residue,
R³⁵ is an alkyl residue with 3 to 6 carbon atoms, which has 2 to n-1 hydroxy groups, where n is the number of carbon atoms, or an alkyl residue with 1 to 6 carbon atoms substituted with 1 to 3 carboxyl groups, aryl residue with 6 to 9 carbon atoms or aralkyl residue with 7 to 15 carbon atoms, or a residue of general formula IIId or IIIe

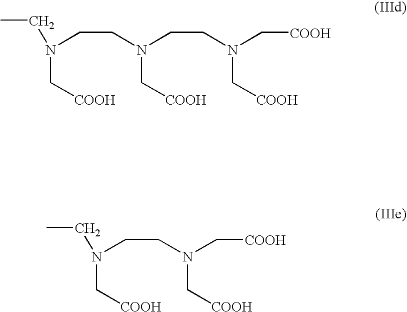

provided that q stands for 1,
or denotes a non-specifically binding macromolecule, R²⁰ and R²¹, R²¹ and R²², R²² and R²³, R²⁴ and R²⁵, R²⁵ and R²⁶, R²⁶ and R²⁷ form, together with the carbon atoms positioned between them, a 5- or 6-membered aromatic or saturated fused ring,
and the physiologically compatible salts thereof.

Formula 8: General structure of the carbocyanines

In the case of the carbocyanines, reference is made to applications DE 4445065 and DE 69911034, the contents of which are to be incorporated in this application.

Unexpectedly, anionic, readily water-soluble substances such as certain carbocyanines can be stably enclosed in the hydrophobic polymer matrix of the nanoparticles described.

In the sense of the invention, an anionic water-soluble substance is encapsulated in a sparingly water-soluble polymer matrix by nanoprecipitation by means of ionic complexing and co-precipitation with a cationic polymer, with formation of particles of a defined size.

By incorporating an NIR-active fluorescent dye in the polymer matrix of the particles, the latter can be detected from the fluorescence by optical imaging non-invasively in the tissue. It thus becomes possible to detect, in vivo, the distribution or accumulation of fluorescence-labeled nanoparticles.

In an especially preferred embodiment, the carbocyanine dye comprises the readily water-soluble anionic tetrasulfocyanine (TSC) (Formula 9).

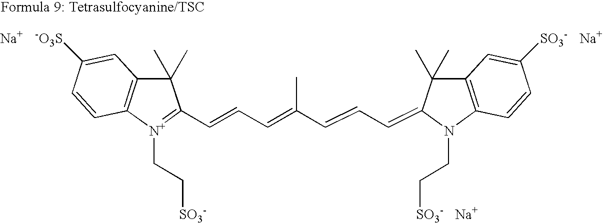

In another especially preferred embodiment, the carbocyanine dye comprises IDCC (indodicarbocyanine) (Formula 10).

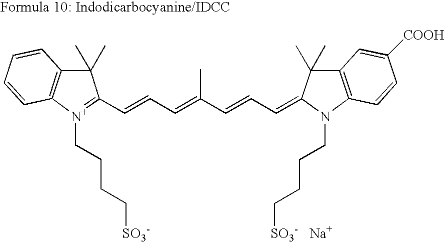

In yet another especially preferred embodiment, the carbocyanine dye comprises ICG (Indocyanine Green) (Formula 11).

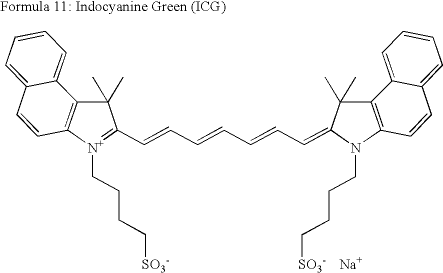

In one embodiment, the encapsulated pharmaceutically active substance comprises a therapeutic agent.

In a preferred embodiment, the therapeutic agent comprises a substance for the treatment of neoplastic diseases, in particular vascularized tumors and metastases, or diseases with inflammatory reactions. The latter may comprise, for example, diseases of the rheumatic morphological class, e.g. rheumatoid arthritis, psoriatic arthritis, collagenoses, vasculitis and infectious arthritides. Other diseases with inflammatory processes and possible tissue changes are chronically inflammatory intestinal diseases (Crohn's disease, ulcerative colitis), multiple sclerosis, atopic dermatitis as well as certain erythema diseases. The following groups in particular may be mentioned as therapeutic substances: immunogenic peptides or proteins, chemotherapeutic agents, toxins, radiotherapeutic agents, radiosensitizers, angiogenesis inhibitors and antiinflammatory substances such as NSAIDs or a combination thereof.

The therapeutic agents for the treatment of neoplastic diseases can be selected from the group comprising the alkylating agents, in particular bendamustine, busulfan, carmustine, chlorambucil, cyclophosphamide, ifosfamide, lomustine, melphalan, nimustine, thiotepa, treosulfan and trofosfamide, the group comprising the antimetabolites, in particular cytarabine, fludarabine, fluorouracil, gemcitabine, mercaptopurine, methotrexate and tioguanine, the group comprising the alkaloids and diterpenes, in particular vinblastine, vincristine, vindesine, vinorelbine, etoposide, and docetaxel, paclitaxel, the taxans group, the group of antibiotics, in particular aclarubicin, dactinomycin, daunorubicin, doxorubicin, epirubicin, idarubicin, mitomycin, mitoxantrone, the group of platinum compounds, in particular carboplatin and cisplatin, the group of the hormones and hormone agonists, in particular testolactone, fosfestrol, tamoxifen, cyproterone flutamide, buserelin, gonadorelin, goserelin, leuprorelin, nafarelin, triptorelin and octreotide and the group of the VEGF inhibitors.

In another preferred embodiment, the therapeutic agent comprises substances that are suitable for the treatment of inflammations. They are selected in particular from the group of nonsteroidal antirheumatic agents (NSAR), in particular salicylates (including acetylsalicylic acid), arylacetic acid derivatives (including acemetacin, diclofenac), propionic acid derivatives (including ibuprofen, ketoprofen, naproxen, tiaprofen), indole derivatives (including indometacin, acemetacin, lonazolac, proglumetacin), oxicams (including piroxicam, tenoxicam), alkalones (including nabumetone), pyrazolones (including azapropazone, pyrazinobutazone, phenylbutazone, oxyphenbutazone), anthranilic acid derivatives (including mefenamic acid, niflumic acid), COX 2 inhibitors (including meloxicam, celecoxib, rofecoxib) and a combination of NSAR and other medicinal products (including the combination of diclofenac and misoprostol), the glucocorticoid group, in particular betamethasone, budesonide, cloprednol, cortisone, deflazacort, dexamethasone, fluocortolone, hydrocortisone, methylprednisolone, prednisolone, prednisone, prednylidene, triamcinolone, beclometasone, flunisolide, fluticasone, alclometasone, amcinonide, clobetasol, clobetasone, clocortolone, desonide, desoximetasone, diflorasone, diflucortolone, fludroxycortide, flumetasone, fluocinolone, fluocinonide, fluocortin, fluprednidene, halcinonide, halometasone, mometasone, prednicarbate, fluorometholone, medrysone, cortisol (hydrocortisone), amcinonide, rimexolone, the group of long-acting antirheumatic agents, in particular chloroquine, hydroxychloroquine, sulfasalazine (salazosulfapyridine), adalimumab, anakinra, etanercept, infliximab, D-penicillamine, the group of immunosuppressants, in particular azathioprine, methotrexate, mycophenolate mofetil, cyclosporin A, cyclophosphamide, chlorambucil and leflunomide, and the group of the antibiotics, in particular the penicillins (including ampicillin, piperacillin, amoxicillin clavulanic acid, tazobactam, apalcillin, penicillin G, oxacillin, flucloxacillin, mezlocillin, phenoxymethylpenicillin), the cephalosporins (including cefotaxime, cefoxitin, cefotiam, cefepime, ceftazidime, ceftriaxone, cefuroxime, cefamandole, cefazolin), the carbapenems (including imipenem, carbapenem, cilastin, meropenem), the quinolones (including moxifloxacin, levofloxacin, ciprofloxacin), the aminoglycosides (including gentamicin, amikacin, netilmicin sulfate, tobramycin, gentamicin), the macrolides (including azithromycin, clarithromycin, erythromycin, roxithromycin), the monobactams (including aztreonam), the glycopeptides (including vancomycin, teicoplanin) and other antibiotics (including doxycycline, clindamycin, ofloxacin, chloramphenicol, amphotericin B, flucytosine, metronidazole, fusidic acid, fosfomycin).

In a preferred embodiment, the polymer nanoparticles comprise precipitated aggregates, which are produced by nanoprecipitation.

For this, the following methods of production are available in particular:
Direct precipitation in a test tube by adding the dissolved mixture of polymeric substances to an aqueous solution containing surfactant, and then mixing thoroughly using a magnetic stirrer.
Precipitation of the mixture of polymeric substances in the aqueous solution containing surfactant by combining the two solutions using a micro-mixer system.
Use of ultrasound for uniform distribution of the mixture of polymeric substances in the aqueous solution containing surfactant.

In the production of nanoparticles by nanoprecipitation, the organic solvent is removed suddenly from the matrix polymer and the substances dissolved with it, if the polymer-containing organic solution is added to a much larger volume of an aqueous solution. Surprisingly, compounds with amino groups (both water-soluble and water-insoluble) that are dissolved in the polymer phase are co-encapsulated in the sparingly soluble polymer during precipitation. Necessary conditions are complete miscibility of the organic solvent (e.g. acetone, ethanol) with water, and insolubility of the matrix polymer in the aqueous phase.

In a preferred embodiment, for all the preceding polymer nanoparticles, the surface of the polymer nanoparticles is modified electrostatically.

The electrostatic modification of the cationic nanoparticle surface is an outstanding advantage of the present invention. On the basis of ionic interactions, the particle surface can be modified with a suitable substance without a chemical coupling reaction. A necessary condition for this is that the modifying agent partially has charges that are opposite to the particle surface charge. This method (electrostatic surface modification by charge titration) permits simple, flexible and versatile modification of the particle surface. Additionally, it is possible to adsorb unstable active substances on the particle surface, and they are thus protected against degradation by enzymes and can accordingly produce a greater therapeutic effect.

A precondition for accumulation (active and passive targeting) of nanoparticles from the bloodstream in the target tissue is that the particles circulate in the bloodstream for a sufficient length of time. According to the invention, by means of the surface modification described above, the circulation time in the body can be adapted individually, in particular by using polyethylene oxides or polyethylene glycols (see Example 5).

A further outstanding advantage is that the electrostatic surface modification described here can be carried out quickly and without any problems directly before use. This is achieved by simple mixing of suitable amounts of the nanoparticle dispersion with the modifying agent.

It is therefore additionally possible to produce and store the core particle separately from the surface modifying agent. On the one hand this is especially advantageous for long-term colloidal stability. On the other hand, extremely labile surface modifying substances like peptides or antibodies can be stored under suitable conditions until they are used.

The separation of core particle and modifying agent also permits surface modification according to the patient's individual requirements. Surface modification based on a modular principle then offers maximum flexibility for diagnosis, therapy and monitoring, with modification being carried out easily, directly by the user.

A preferred structure of the surface-modifying agent for cationically functionalized polymer nanoparticles, in particular the PBCA nanoparticles described, is shown in Formula 5. The partially anionically charged moiety fulfils the function of an anchorage for the positively charged particle surface through electrostatic interactions. The neutral moiety directed toward the surrounding aqueous medium comprises polyethylene glycol and/or polyethylene oxide units (PEG units) of varying length. PEG chains with a molecular weight of 100 to 30000 dalton are preferred, and those with 3000 to 5000 dalton are especially preferred. This moiety can alternatively also comprise other suitable structures, e.g. hydroxyethyl starch (HES) and all possible polymeric compounds thereof. Residue R is preferably hydrogen or a methyl unit.

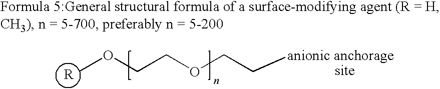

In an especially preferred embodiment, the surface of the polymer nanoparticle is modified with Glu(10)-b-PEG(110) (Formula 6). The carboxylate groups of the glutamic acid subunits of the block copolymer serve as the negative moiety (anchorage site).

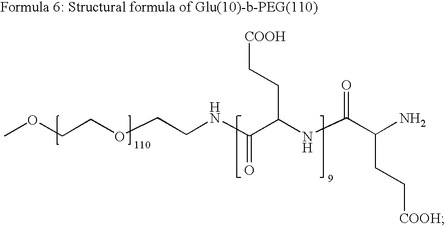

In an especially preferred embodiment, a target structure is present.

This target structure possesses at least one negatively charged moiety, which is applied to the cationic particle surface by electrostatic interactions.

Another especially preferred structure of the surface-modifying agent for cationically functionalized polymer nanoparticles, in particular the PBCA nanoparticles described, is shown in Formula 7.

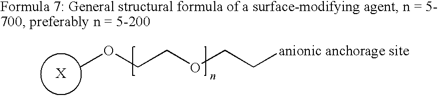

The partially anionically charged moiety fulfils the function of an anchorage site on the positively charged particle surface by electrostatic interactions. The central, neutral moiety comprises polyethylene glycol units and/or polyethylene oxide units (PEG units) of varying length. PEG chains with a molecular weight of 100 to 30000 dalton are preferred here, and those with 3000 to 5000 dalton are especially preferred. This moiety can alternatively also comprise other suitable structures, e.g. hydroxyethyl starch (HES) and all possible polymeric compounds thereof.

Ligand X of the surface-modifying agent, also called target structure hereinafter, is for improving passive and active accumulation mechanisms of the polymer nanoparticles.

Suitable ligands as target structures can be antibodies, peptides, receptor ligands of ligand mimetics or an aptamer. The following may be considered as structures: amino acids, peptides, CDRs (complementary determining regions), antigens, haptens, enzymatic substances, enzyme cofactors, biotin, carotinoids, hormones, vitamins, growth factors, lymphokines, carbohydrates, oligosaccharides, lecithins, dextrans, lipids, nucleosides for example native, modified or artificial nucleosides containing a DNA or an RNA molecule, nucleic acids, oligonucleotides, polysaccharides, B-, A-, Z-helix or hairpin structure, a chemical unit, modified polysaccharides as well as receptor-binding substances or fragments thereof. Target structures can also be transferrin or folic acid or parts thereof or all possible combinations of the aforementioned.

According to the invention, these ligands are bound to the nanoparticles by electrostatic interactions, but it is also possible for the ligands to be bound to the particle surface via covalent bonds. It is further possible to incorporate a linker between ligand and nanoparticle.

Electrostatic attachment of the target structures takes place by charge interactions with at least one negatively charged moiety on the cationic particle surface. Especially compounds, especially groups such as acetate, carbonate, citrate, succinate, nitrate, carboxylate, phosphate, sulfonate or sulfate groups, and salts and free acids of these groups, are suitable as negatively charged moiety (anchorage site).

In one embodiment, the size of the nanoparticles is between 1 nm and 800 nm.

In a preferred embodiment, the size of the nanoparticles is between 5 nm and 800 nm.

In a preferred embodiment, the size of the nanoparticles is between 1 nm and 500 nm.

In a preferred embodiment, the size of the nanoparticles is between 1 nm and 300 nm.

In an especially preferred embodiment, the size of the nanoparticles is between 5 nm and 500 nm.

In yet another especially preferred embodiment, the size of the nanoparticles is between 5 nm and 300 nm.

In yet another especially preferred embodiment, the size of the nanoparticles is between 10 nm and 300 nm.

The size of the resultant polymer nanoparticles is determined by photon correlation spectroscopy (PCS).

In an especially preferred embodiment, the polymer nanoparticles are characterized by the execution of the following process steps:
The water-insoluble polymer is dissolved in a suitable organic solvent that is completely miscible with water, preferably acetone, methanol, ethanol, propanol, dimethylsulfoxide (DMSO), or in a mixture of these solvents with water.
The cationic polymer is dissolved in a suitable solvent that is completely miscible with water, preferably acetone, methanol, ethanol, propanol, dimethylsulfoxide (DMSO), or in a mixture of these solvents with water.
The active substance (diagnostic agent or therapeutic agent) is dissolved in an organic solvent that is completely miscible with water, preferably acetone, methanol, ethanol, propanol, dimethylsulfoxide (DMSO), or in a mixture of these solvents with water.
A completely homogeneous solution is produced from cationic polymer, water-insoluble polymer and active substance.
Adding the dissolved mixture of polymeric substances to a surfactant-containing solution, in particular with Pluronic F68, Triton X-100 and Synperonic T707 as surfactant, brings about the spontaneous formation of a colloidal precipitated aggregate.
The organic solvent is then removed completely either at atmospheric pressure or reduced pressure, by lyophilization or by heating, or other suitable methods.
For modifying the particle surface, the aqueous, stable nanoparticle dispersion is mixed in suitable proportions with the modifying agent dissolved in water. The appropriate proportions are determined by stepwise titration of the particle dispersion with the modifying agent. The extent of electrostatic surface modification (charge titration) is monitored by determining the zeta potential.

The nanoparticles described can be processed further, using suitable pharmaceutical excipients, to various pharmaceutical forms, which are suitable for administration to humans or animals. These include in particular aqueous dispersions, lyophilizates, solid oral pharmaceutical forms such as quick-dissolving tablets, capsules and others. Suitable pharmaceutical excipients can be: sugar alcohols for lyophilization (e.g. sorbitol, mannitol), tableting aids, polyethylene glycols etc.

The aqueous nanoparticle dispersion or a further developed pharmaceutical form can be applied by the oral, parenteral (intravenous), subcutaneous, intramuscular, intraocular, intrapulmonary, nasal, intraperitoneal or dermal route and by all other possible routes of administration for humans or animals.

The invention relates to a method of production of a polymer nanoparticle, characterized in that the following process steps are carried out:
Dissolution of the cationic polymer in an organic solvent or a solvent/water mixture
Dissolution of the water-insoluble polymer in an organic solvent
Dissolution of the active substance (diagnostic agent or therapeutic agent) in an organic solvent or a solvent/water mixture,
Preparation of a completely dissolved mixture of cationic polymer, water-insoluble polymer and active substance
Adding the mixture to a surfactant-containing solution, with spontaneous formation of precipitated aggregates,
Removal of the solvent.
Electrostatic surface modification of the particles by adding together the nanoparticle dispersion and modifying agent in suitable amounts (optional).

### DEFINITIONS

The term “active substance”, as used here, comprises therapeutically and diagnostically active compounds. It also comprises compounds that are active in animals other than humans and in plants.

The term “matrix polymer”, as used here, describes the polymer that forms the quantitatively greater part of the particle mass, it being possible for other encapsulated substances (both any required additives and pharmaceutically active substances) to be encapsulated uniformly and/or nonuniformly.

The term “(nano)-precipitation”, as used here, describes the formation of a colloidal precipitate by precipitation of a sparingly water-soluble polymer on being introduced into an aqueous phase, with thorough mixing of the solvents.

In the case of co-precipitation, there is simultaneous precipitation of several substances, which in the sense of the invention can be both water-soluble and sparingly water-soluble.

A “precipitated aggregate”, as used here, arises in the course of nanoprecipitation. This precipitated aggregate comprises, according to the invention, a matrix polymer, in which other polymeric substances as well as pharmaceutically active substances can be encapsulated partially or completely. There may be uniform or nonuniform distribution of the co-encapsulated substances in the matrix polymer.

An “anchorage site”, as used here, describes an ionic moiety of the modifying agent, which permits the immobilization and thus localization of the modifying agent on the charged particle surface by ionic interactions between oppositely charged compounds.

“Charge titration” describes the process of electrostatic coupling of the anchorage site on the particle surface, which is accomplished using measurement of the zeta potential. The charged anchorage site then alters the zeta potential of the particle to the charge of the anchorage site.

“Surfactants” in the sense of the invention are, on the one hand, surface active substances that lower the interfacial tension between two immiscible phases, so that stabilization of colloidal dispersions becomes possible. Furthermore, surfactants according to the invention can be substances of any kind that are able to stabilize colloidal dispersions sterically and/or electrostatically.

The term “active targeting” is used when tissue-specific or cell-specific ligands are employed for targeted accumulation. Active ligands can be coupled both to active substances directly (ligand/active substance conjugates) and to the surface of colloidal vehicle systems.

The term “passive targeting” is used when the active substance is distributed as a result of (nonspecific) physical, biochemical or immunological processes. The enhanced permeation and retention effect (EPR effect) is considered to be primarily responsible for this. It is a mechanism of passive accumulation, which makes use of the structural peculiarities of tumoral or of inflamed tissue [Ulbrich K., Subr V., Adv. Drug Deliv. Rev., 2004; 56(7): 1023-1050].

The term “surface potential”, also called surface charge, is equivalent to the term “zeta potential”. The zeta potential is determined by laser Doppler anemometry (LDA).

The surface potential, also called zeta potential, denotes the potential of a migrating particle on the shear plane, i.e. when as a result of movement of the particle most of the diffuse layer has been sheared off. The surface potential was determined by laser Doppler anemometry using a “Zetasizer 3000” (Malvern Instruments).

The migration velocity of the particles in the electric field is determined by laser Doppler anemometry. Particles with a charged surface migrate in an electric field toward the oppositely charged electrode, the migration velocity of the particles being a function of the amount of surface charges and the applied field strength. For determination of the migration velocity, particles migrating in the electric field are irradiated with a laser and the scattered laser light is detected. Owing to the movement of the particles, a frequency shift is measured in the reflected light in comparison with the incident light. The magnitude of this frequency shift depends on the migration velocity and is called the Doppler frequency (Doppler effect). The migration velocity of a particle can be found from the Doppler frequency, the scattering angle and the wavelength. The electrophoretic mobility is found from the quotient of the migration velocity and the electric field strength. The electrophoretic mobility multiplied by a factor of 13 corresponds to the zeta potential, with unit [mV].

The measurements (n=5) were performed with a Zetasizer Advanced 3000 and a Zetamaster from the company Malvern Instruments Ltd. (Worcestershire, England) after dilution in a dispersion medium with low electrolyte content (MilliQ water: resistance value 18.2 mΩ.cm, 25° C. and TOC content (total organic carbon)<10 ppb) and at a defined pH value (pH 6.8-7.0). The software used was PCS V1.41/PCS V1.51 Rev. Control measurements of the zeta potential were carried out with latex standard particles from the company Malvern Instruments Ltd. (−50 mV±5 mV). The measurements were performed with the standard settings of the company Malvern Instruments Ltd.

The size of the nanoparticles was determined by dynamic light scattering (DLS) using a “Zetasizer 3000” (Malvern instruments). In addition, micrographs were obtained in the scanning electron microscope (SEM), and an example is shown in FIG. 12. FIG. 12 also confirms the spherical shape of the nanoparticles.

Determination of particle size by DLS is based on the principle of photon correlation spectroscopy (PCS). This method is suitable for the measurement of particles with a size in the range from 3 nm to 3 μm. In solution, the particles are subject to random motion, caused by collision with liquid molecules of the dispersion medium, the driving force of which is the Brownian motion of the molecules. The resultant motion of the particles is faster, the smaller the particle diameter. If a sample in a cuvette is irradiated with laser light, scattering of the light occurs at the randomly moving particles. Owing to this motion of the particles, the scattering is not constant, but fluctuates over time. The fluctuations in intensity of the scattered laser light detected at an angle of 90° are greater for faster moving, and hence smaller, particles. On the basis of these variations in intensity, the particle size can be concluded by means of an autocorrelation function. The mean particle diameter is calculated from the decrease in the correlation function. For correct calculation of the mean particle diameter, the particles should be of spherical shape, which can be verified with SEM micrographs (see above), and they should not sediment, nor float to the surface. The measurements were carried out with samples at suitable dilution, at a constant temperature of 25° C. and a specified viscosity of the solution. The measuring instrument was calibrated with standard latex particles of varying size from the company Malvern Instruments Ltd.

The scanning electron micrographs (SEM micrographs) for determining particle size were obtained with a field emission scanning electron microscope of type XL-30-SFEG from the company FEI (Kassel, Germany). The samples were sputtered beforehand with a 5 nm gold-palladium film in a high-vacuum Sputter 208 HR from the company Cressington (Watford, England).

The solubility of a substance states whether, and to what extent, a pure substance can be dissolved in a solvent. It thus characterizes the property of a substance, to mix with the solvent with homogeneous distribution (as atoms, molecules or ions). The solubility of a compound is defined as the concentration of a saturated solution that is in equilibrium with the undissolved sediment as a function of the temperature (room temperature). A sparingly soluble compound has a solubility <0.1 mol/l, a moderately soluble one between 0.1-1 mol/l and a readily soluble compound >1 mol/l.

The invention will now be described further in the examples given hereunder, without being limited to them.

### EXAMPLES

### Example 1

### Production of PBCA by Anionic Polymerization

Sicomet 6000 is used for PBCA production by anionic polymerization of butylcyanoacrylate (BCA). The polymerization process is carried out by slow, permanent dropwise addition of a total of 2.5% [w/v] BCA to a 1% [w/v] Triton X-100 solution at pH 2.2. The pH value is adjusted beforehand by means of a 0.1N HCl solution. The resultant dispersion is stirred at a constant 450 rev/min while cooling on an ice bath (approx. 4° C.) for 4 hours. Then larger agglomerates are removed by filtration through a pleated paper filter. By adding ethanol, the BCA polymerized to PBCA is precipitated and the filter residue obtained from it is washed several times with purified water (MilliQ system). After drying the PBCA filter residue in a drying cabinet at 40° C. for 24 h, an average molecular weight is determined by GPC (Mn ˜2000 Da). Polysterol standards are used.

### Example 2

### Production of Functionalized PBCA Nanoparticles by Nanoprecipitation

i) PBCA-P(DMAEMA) Nanoparticles

500 μl of a 2% acetone PBCA solution [w/v] is mixed thoroughly with 100 μl of a 2% acetone P(DMAEMA) solution [w/v] in closed conditions (to prevent evaporation of the acetone) using a standard laboratory shaker. The PBCA used for this is prepared according to Example 1. 100 μl of each of the dye solutions described in the following is added to this polymer mixture.

Dye solution a: 3 mg of Indocyanine Green is first dissolved in 300 μl of purified water in the ultrasonic bath, and then 700 μl acetone is added.

Dye solution b, c, d: The dyes DODC, IDCC and Coumarin 6 are used in a 0.02% acetone solution [w/v].

The thoroughly mixed dye-polymer mixture is taken up in a 2.5 ml Eppendorf pipette and pipetted into 10 ml of a vigorously stirred 1% [w/v] Synperonic T707 solution. The nanoparticle dispersion is stirred for 2 h at 600 rev/min (standard magnetic stirrer) and for a further 16 h at 100 rev/min for complete evaporation of the solvent. It is processed by centrifugation in Eppendorf-Caps. In each case 1 ml of the particle dispersion and 0.5 ml of a 1% [w/v] CETAC solution (cetyltrimethylammonium chloride solution) are mixed thoroughly and centrifuged for 10 min at 14000 rev/min (in a Sigma 2 K 15 laboratory centrifuge). The supernatant is removed, the particles are redispersed in 1% CETAC solution and centrifuged again. This washing process is repeated three times, then finally the particles are taken up in a 1% solution of Synperonic T707.

ii) PBCA-[PEI-IDCC] Nanoparticles

500 μl of a 2% acetone PBCA solution [w/v] is used with PEI 1.8 kDa in isopropanol (2% [w/v]). 100 μl of each of the dye solutions a-d stated in i) is used.

The thoroughly mixed dye-polymer mixture is taken up in a 2.5 ml Eppendorf pipette and pipetted into 10 ml of a vigorously stirred 1% Triton X-100 solution. The nanoparticle dispersion is stirred for 2 h at 600 rev/min (standard magnetic stirrer) and for a further 16 h at 100 rev/min for complete evaporation of the solvent. It is processed by centrifugation in Eppendorf-Caps. In each case 1 ml of the particle dispersion and 0.5 ml of a 1% [w/v] CETAC solution (cetyltrimethylammonium chloride solution) are mixed thoroughly and centrifuged for 10 min at 14000 rev/min (in a Sigma 2 K 15 laboratory centrifuge). The supernatant is removed, the particles are redispersed in the 1% CETAC solution and centrifuged again. This washing process is repeated three times, then finally the particles are taken up in a 1% solution of Triton X-100.

iii) PLGA-P(DMAEMA) Nanoparticles

500 μl of a 2% acetone PLGA solution [w/v] is used with 100 μl P(DMAEMA) in acetone (2% [m/v]). 100 μl of each of the dye solutions a-d stated in i) is used. The thoroughly mixed dye-polymer mixture is taken up in a 2.5 ml Eppendorf pipette and pipetted into 10 ml of a vigorously stirred 1% Synperonic T707 solution. The nanoparticle dispersion is stirred for 2 h at 600 rev/min (standard magnetic stirrer) and for a further 16 h at 100 rev/min for complete evaporation of the solvent. It is processed by centrifugation in Eppendorf-Caps. In each case 1 ml of the particle dispersion and 0.5 ml of a 1% [w/v] CETAC solution (cetyltrimethylammonium chloride solution) are mixed thoroughly and centrifuged for 10 min at 14000 rev/min (in a Sigma 2 K 15 laboratory centrifuge). The supernatant is removed, the particles are redispersed in 1% CETAC solution and centrifuged again. This washing process is repeated three times, then finally the particles are taken up in a 1% solution of Synperonic T707.

### Example 3

### Influencing Nanoprecipitation by Varying the Polymer Content in the Surfactant Phase

It is shown in FIG. 1 that the particle size of the PBCA-P(DMAEMA) nanoparticles can be controlled during production by varying the polymer concentration. PBCA-P(DMAEMA) nanoparticles produced according to Example 2 are stabilized with the surfactant Synperonic T707. During particle production (nanoprecipitation), the volume of the organic polymer solution injected into the surfactant phase is kept constant and only the polymer concentration is varied correspondingly. All the other production conditions (surfactant concentration, ratio of polymers PBCA:P(DMAEMA)=10:1, dye concentration, temperature, stirring speed/magnetic stirring bar, vessel, type of injection) remain constant.

The use of a lower polymer concentration in the surfactant phase during precipitation leads to smaller particle diameters. Over the test period, no change in particle size was found at equal polymer content.

### Example 4

### Cationically Functionalized Particles

The cationically functionalized particles are prepared according to Example 2. FIG. 2 shows the particle diameter as well as the zeta potential of PBCA-[PEI-IDCC] nanoparticles, which are stabilized either by the surfactant Triton X-100 or Pluronic F 68. Owing to encapsulation of the polycation polyethyleneimine in the PBCA-matrix, the particles have a positive zeta potential between 30 mV and 40 mV. Both the particle size and the zeta potential are constant before and after processing of the particles (washing process)—proof of good stability of the particles.

### Example 5

### Electrostatic Surface Modification of PBCA-[PEI-IDCC] Nanoparticles with Glu(10)-b-PEG(110)

The PBCA-[PEI-IDCC] nanoparticles used here are prepared according to Example 2.

For modifying the particle surface the stable aqueous nanoparticle dispersion is mixed in suitable proportions with the modifying agent dissolved in water. The appropriate proportions are determined by stepwise titration of the particle dispersion with the modifying agent. The extent of electrostatic surface modification (charge titration) is monitored by determining the zeta potential. FIG. 3 shows the variation in zeta potential from +25 mV to approx. −30 mV by stepwise addition of the modifying agent (Glu(10)-b-PEG(110)) to the particle dispersion (charge titration).

### Example 6

### SEM Micrographs of PBCA-P(DMAEMA) Nanoparticles loaded with various Fluorescence Dyes

The PBCA-P(DMAEMA) loaded with the dyes diethyloxadicarbocyanine (DODC) and Coumarin 6 are prepared according to Example 2.

FIG. 4 shows an SEM micrograph of DODC-loaded PBCA-P(DMAEMA) nanoparticles.

FIG. 5 shows an SEM micrograph of Coumarin 6 loaded PBCA-P(DMAEMA) nanoparticles.

### Example 7

### Cell Culture Tests

The HeLa cell line is cultivated in 225 cm² culture flasks at 37° C. and 5% CO₂ in Dulbecco's Modified Eagles Medium (DMEM) with addition of 10% fetal calf serum (FCS) and 2 mM L-glutamine. No additions of antibiotic (penicillin/streptamycin) were used, so as to influence the cell processes as little as possible. The cells are passaged regularly and seeding for test purposes is carried out 24 h before the start of the investigations. For the investigations, the cells are seeded in 96-well plates from the company Falcon/Becton Dickinson.

A visual check on the vitality or typical morphology of the cells is carried out before starting the tests. Then the FCS-containing medium is drawn off and replaced with 50 μl of serum-free medium.

After a nanoparticle dispersion, prepared according to Example 2, has been incubated for a maximum of 60 minutes, the supernatant particle dispersion is drawn off and the cells are washed with PBS 2-3 times. The dye MitoTracker Red CMXRos from the company Molecular Probes Europe BV, Leiden (NL) (0.25 μl/ml), diluted beforehand in the medium, is used for staining the mitochondria. Incubation with 50 μl of the dye solution is carried out for 15 min in the incubator (37° C., 5% CO₂). Then the dye solution is drawn off and the cells are washed 2-3 times with PBS. The cells are fixed with 100 μl of 1.37% formaldehyde for 10 min at room temperature. After drawing off the fixing solution, the cells are washed 2-3 times with PBS. The cell nuclei are stained in the already fixed cells with Hoechst 33342. For this, 100 μl of the dye solution diluted in PBS (2 μg/ml) is incubated for 10 min at room temperature. After removing the dye solution, the cells are washed with 100 μl PBS 2-3 times. The fixed plates are stored, with 200 μl PBS/well, protected from the light, in the refrigerator at 8° C. until the investigation using fluorescence microscopy.

### Example 8

### Influence of Functionalized Particle Surfaces on Cellular Uptake

TABLE 1
Particle diameter d_{hyd}, polydispersity index and
zeta potential of PBCA-P(DMAEMA) nanoparticles (NP)
loaded with (non)-functionalized Coumarin 6

| | Size d_{hyd} | Polydispersity | Zeta potential |
|---|---|---|---|
| | [nm] | index [PI] | [mV] |
| 1.) Unmodified NP | 191 | 0.13 | +31.5 ± 1.5 |
| 2.) NP with folio acid | 195 | 0.06 | +8.1 ± 3.7 |
| 3.) NP with Glu-PEG | 208 | 0.08 | −28.4 ± 1.2 |

The nanoparticles used in Example 8 are prepared according to Example 2. The particles, unmodified or after electrostatic surface modification with folic acid or Glu(10)-b-PEG(110), have the properties shown in Table 1.

In the 96-well plate used for the test, all the wells have the same cell density (seeding 24 h before the test: 1×10⁴ cells). A constant particle concentration of the particles shown in the table (Table 1) is incubated for a period of 60 minutes in the incubator. Then the cells are washed, fixed, and measured on the next day. The fluorescing cells are photographed with an automatic fluorescence microscope at 20-times magnification and constant exposure time (see FIG. 6). FIG. 6 shows how the cellular uptake behavior is influenced by different surface properties of one and the same nanoparticle charge. Unmodified particles in Row 1.) with a cationic surface potential display higher affinity for the cell surface, as can be seen from the greater fluorescence contrast on or in the cells. The internalization of particles with negative surface potential after titration with Glu(10)-b-PEG(110), which is also effective, can be seen from the enlarged section of the cells from Row 3.).

### Example 9

### Cellular Uptake behavior of Glu(10)-b-PEG(110) Modified PBCA P(DMAEMA) Nanoparticles

The nanoparticles used in Example 9 are prepared according to Example 2. After electrostatic surface modification with Glu(10)-b-PEG(110), the cellular uptake behavior of the particles (d_{hyd}=171 nm; ZP=−33 mV) is investigated.

The brightly fluorescing points, which are endosomes or endolysosomes, are proof of efficient uptake of the nanoparticles into the cell by endocytosis (FIG. 7). The scale of the magnification verifies that in this photograph, individual particles cannot be visible on account of their size of less than 200 nm. The large number of particles inside these vesicles (endosomes/endolysosomes) causes the strong, punctiform fluorescence contrast in the cytoplasm. The cellular uptake of PBCA-P(DMAEMA) nanoparticles surface modified with Glu(10)-b-PEG(110) is shown schematically in FIG. 8.

### Example 10

### Accumulation of Glu(10)-b-PEG(110) Modified PBCA P(DMAEMA) Nanoparticles in the Cell Nucleus

Photographing the mid-plane of the cell by means of the confocal laser scanning microscope (FIG. 9) shows that there is partial accumulation of the particles in the cell nucleus.

### Example 11

### Increased Particle Uptake with Incubation of Higher Particle Concentration

Glu(10)-b-PEG(110) surface modified PBCA-P(DMAEMA) particles, loaded with Coumarin 6, are prepared according to Example 2. A low particle concentration of 0.21 mg/ml (FIG. 10) and a higher particle concentration of 0.85 mg/ml (FIG. 11) were incubated for the same length of time on the cells according to Example 7. FIG. 11 shows, relative to FIG. 10, an increased particle uptake on incubation of a higher particle concentration.

### Example 12

### Characterization of the PBCA-[P(DMAEMA)-ICG] Nanoparticles

The particle size of the surface modified PBCA-[P(DMAEMA)-ICG] nanoparticles used for the animal experiment, over a period of 7 days after production for the animal experiment, is shown (FIG. 13). The constant particle size, and constant low polydispersity index (Pl<0.1) as a characteristic feature of a very narrow particle size distribution, are evidence of good stability of the surface modified particles.

On the basis of the SEM micrograph (FIG. 12), it can additionally be asserted that they are spherical nanoparticles with a size of about 200 nm.

By means of charge titration, the cationic surface of the PBCA-P(DMAEMA) nanoparticles is modified with block copolymer Glu(10)-b-PEG(110) (see FIG. 14). The surface charge, measured as zeta potential, is titrated correspondingly from approx. +30mV beyond the neutral point until dissociation equilibrium is attained at about −30mV. The surface modified PBCA-[P(DMAEMA)-ICG]-particles do not show, over the period investigated of 7 days after titration, any change in the zeta potential. The unchanged particle size and the constant, low PI thus provide evidence of good particle stability.

FIG. 15 shows the UV-Vis absorption spectra of an aqueous ICG solution and of the ICG nanoparticle dispersion (washed and unwashed). Indocyanine Green is a near-infrared fluorescence dye, with absorption and emission spectrum in the wavelength range 650-900 nm. Complexing and encapsulation of ICG by means of the cationic polyacrylate P(DMAEMA) leads to a minimal bathochrome shift of the two wavelength maxima.

### Example 13

### Animal Experiments

The animals used were supplied by the company Taconic M&B. They are female albino nude mice of the type NMRI nude. The fully grown animals have a weight of 22-24 g after approx. 8 weeks. Five female nude mice are inoculated with 2×10⁶ cells of an F9-teratoma in the right hind flank. The cells were obtained from the company ATCC/LGC Promochem GmbH. They are mouse-derived embryonic cells of a testinal teratocarcinoma, which is used as a tumor model for cancer research purposes in mice. After 18 days, in four of the five mice, tumors have grown with an average size of approx. 0.5-1 cm diameter. The animals are anesthetized permanently with a Rompun-Ketavet injection at a dose of 100 μl/10 g animal for the first hour of the experiment. The injection solution comprises a 1:1 mixture of a 1:10 dilution of Rompun or 1:5 dilution of Ketavet with physiological saline. Then 200 μl of the nanoparticle dispersion is injected i.v. in the caudal vein. Subsequent anesthesia is effected with Rompun-Ketavet via the lungs as inhaled anesthetic, for minimal loading of the animals' circulation. In a time frame of 24 and 48 h after injection of the substance, the animals were examined visually by fluorescence.

It can be seen from FIG. 17 that Glu(10)-b-PEG(110)-modified PBCA-[P(DMAEMA)-ICG] nanoparticles, after intravenous application (caudal vein), are able to accumulate in the tumor tissue by passive accumulation mechanisms (EPR effect). Examination of the tumors ex vivo shows definite intensification of the fluorescence contrast for the treated tumor tissue compared with the untreated tumor tissue (compare FIG. 18 b with a, or c with a). Multiple, delayed detection of the fluorescence in one and the same animal is possible after 24 h and 48 h (FIG. 17). Accordingly, the particles can circulate in vivo for a sufficient length of time and thus accumulate in the tumor. The electrostatically pegylated surface is thus bound stably to the particle surface. There is rapid biliary elimination of non-tumor-associated particles from the liver. This is indicated by absence of NIR fluorescence contrast in the liver after 24 or 48 h. Rapid elimination of particles that are not accumulated in the tumor from the organism (e.g. liver) permits good tumor contrast at minimal loading of other organs, a prerequisite for a contrast agent system having little side effect.

The equipment used for the animal experiments was constructed by the company LMTB (Berlin, Germany). It has the following separate components:

Laser: Diode laser (742 nm), model Ceralas PDT 742/1.5W; made by CeramOptec (Bonn, Germany)

Excitation filter: 1×LCLS-750 nm-F; 1×740 nm interference filter (bandpass)

Emission filter: 1×bk-802.5-22-c1; 1×bk-801-15-c1

Camera: Peltier air-cooled CCD camera, model C4742-95 12ER, made by Hamamatsu (Herrsching, Germany)

Software: Simple PCI 5.0, from Compix/Hamamatsu

### FIGURES

FIG. 1: Control of particle diameter by varying the polymer concentration;

FIG. 1 shows that the particle size of the PBCA-P(DMAEMA) nanoparticles can be controlled during production by varying the polymer concentration.

FIG. 2: Particle diameter d_{hyd} and zeta potential of PBCA-[PEI-IDCC]-NP in washed and unwashed particles in two different surfactants (TX-100=Triton X-100, F 68=Pluronic F-68);

The diagram shows both the particle diameter and the zeta potential of PBCA-[PEI-IDCC] nanoparticles, which were stabilized either by the surfactant Triton X-100 or Pluronic F 68.

FIG. 3: Zeta potential of Glu(10)-b-PEG(110) modified PBCA-[PEI-IDCC] nanoparticles;

This figure shows the change in zeta potential from +25 mV to approx. −30 mV by stepwise addition of the modifying agent (Glu(10)-b-PEG(110)) to the particle dispersion (charge titration).

FIG. 4: SEM (scanning electron microscope) photograph of DODC-loaded PBCA-P(DMAEMA) nanoparticles;

This figure shows an SEM micrograph of DODC-loaded PBCA-P(DMAEMA) nanoparticles.

FIG. 5: SEM micrograph of Coumarin 6-loaded PBCA-P(DMAEMA) nanoparticles;

The figure shows an SEM micrograph of Coumarin 6-loaded PBCA-P(DMAEMA) nanoparticles.

FIG. 6: Effect of functionalized particle surfaces on cellular uptake:

a) comparison of cellular uptake behavior after surface modification; row 1: unmodified particles; row 2: NP with folic acid; row 3: NP with Glu(10)-b-PEG(110);

b) detail: row 3/well 1/site 15; arrows indicate definite intensification of fluorescence in the cell nucleus.

FIG. 7: Nanoparticle uptake in HeLa cells; fluorescence of the nanoparticles as gray-scale image;

The figure shows the cellular uptake behavior of Glu(10)-b-PEG(110) modified PBCA P(DMAEMA) nanoparticles in HeLa cells.

FIG. 8: Schematic representation of cellular uptake of PBCA-P(DMAEMA) nanoparticles surface modified with Glu(10)-b-PEG(110);

Abbreviations used=PEG-NP: pegylated coumarin-containing PBCA-P(DMAEMA) nanoparticles; NP: coumarin-loaded PBCA-P(DMAEMA) nanoparticles; CP: clathrin-coated pits; ES: endosomes; LS: lysosomes; ELS: endolysosomes; ZK: cell nucleus; H+: H+ATPase; PEG-Glu: free Glu(10)-b-PEG(110) block copolymer; size relations do not correspond to reality.

FIG. 9: a) representation of fluorescence in the cell mid-plane (CLSM, confocal scanning laser microscope), b) computer-based 3D-representation of fluorescence;

The illustration shows the accumulation of the Glu(10)-b-PEG(110) modified PBCA-P(DMAEMA) nanoparticles in the cell nucleus. This is possible through loading with the fluorescence-active dye Coumarin 6.

FIG. 10: Reduced particle uptake in incubation of the lower particle concentration: 0.21 mg/ml; fluorescence of the NPs as gray-scale image;

The figure shows fluorescing HeLa cells after incubating a particle concentration of 0.21 mg/ml. Glu(10)-b-PEG(110) surface modified PBCA-P(DMAEMA) particles were used.

FIG. 11: Increased particle uptake in incubation of higher particle concentration: 0.85 mg/ml; fluorescence of the NPs as gray-scale image;

The figure shows much more strongly fluorescing HeLa cells after incubating a higher particle concentration of 0.85 mg/ml. Glu(10)-b-PEG(110) surface modified PBCA-P(DMAEMA) particles were used.

FIG. 12: SEM micrograph of PBCA-[P(DMAEMA)-ICG] nanoparticles

FIG. 13: Particle diameter d_{hyd} of the PBCA-[P(DMAEMA)-ICG] nanoparticles, surface modified with Glu(10)-b-PEG(110);

This shows the particle size of the surface modified PBCA-[P(DMAEMA)-ICG] nanoparticles used for the animal experiments over a period of 7 days after production for the animal experiments.

FIG. 14: Zeta potential of the untitrated (washed/unwashed) and the titrated PBCA [P(DMAEMA) ICG] nanoparticles;

The figure shows the surface charge, measured as zeta potential, of the PBCA-P(DMAEMA) nanoparticles modified with the block copolymer Glu(10)-b-PEG(110). This was titrated correspondingly from approx. +30 mV through and beyond the neutral point up to attainment of dissociation equilibrium at about −30 mV.

FIG. 15: UV-Vis absorption spectra: a) aqueous ICG solution, b) PBCA-[P(DMAEMA)-ICG] NP, unwashed; c) PBCA-[P(DMAEMA)-ICG] nanoparticles, washed;

This figure shows the UV-Vis absorption spectra of an aqueous ICG solution and of the ICG-nanoparticle dispersion (washed and unwashed).

FIG. 16: Emission spectrum of the PBCA-[P(DMAEMA)-ICG] nanoparticles and of an aqueous ICG solution;

The figure shows the corresponding emission spectra of the aqueous ICG solution compared with the nanoparticle dispersion.

FIG. 17: Detection of NIR fluorescence in vivo;

The diagrams show the NIR fluorescence in a time frame of 24 and 48 h after injection of the substance (a) 24 h ventrally, b) 24 h laterally, c) 48 h laterally, d) blank value, ventrally).

FIG. 18: NIR fluorescence contrast of the tumor tissue ex vivo 48 h after treatment;

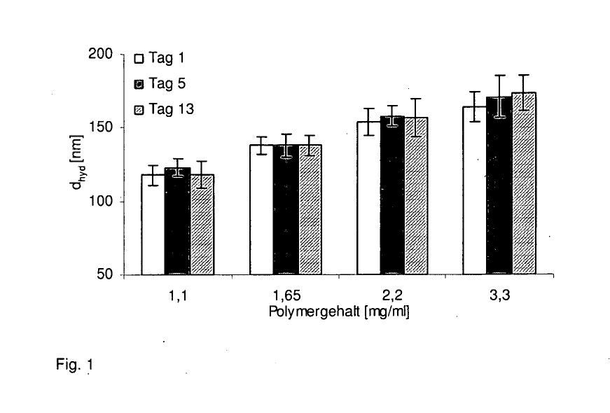

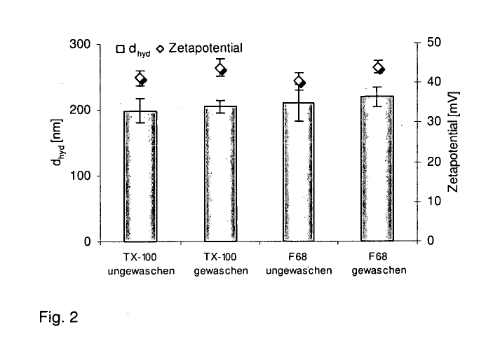

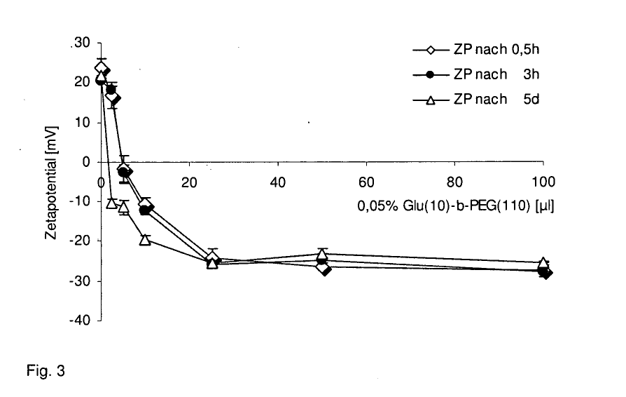

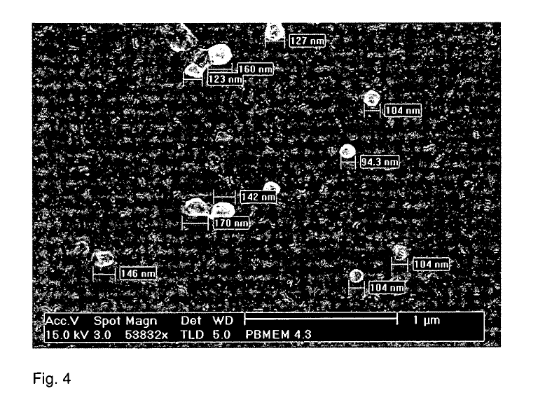

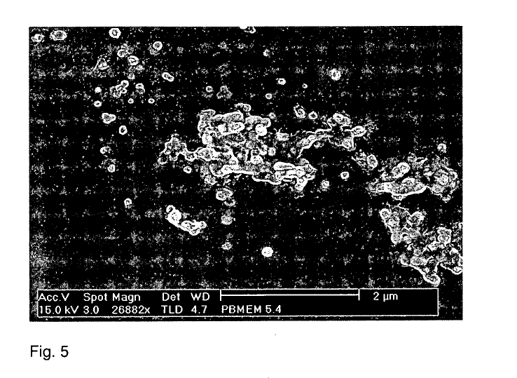

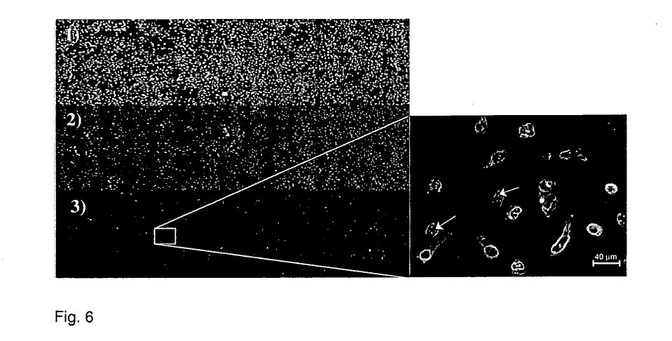

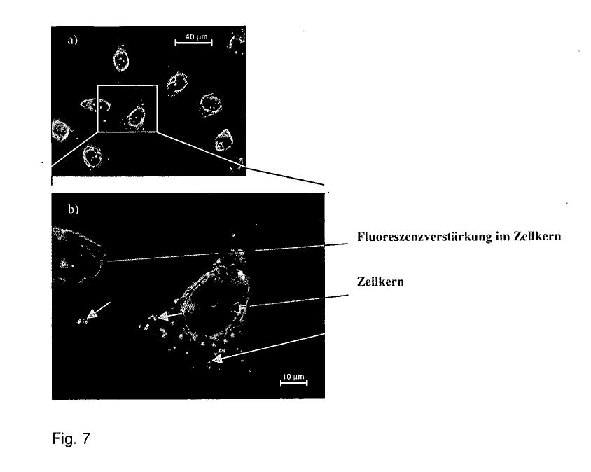

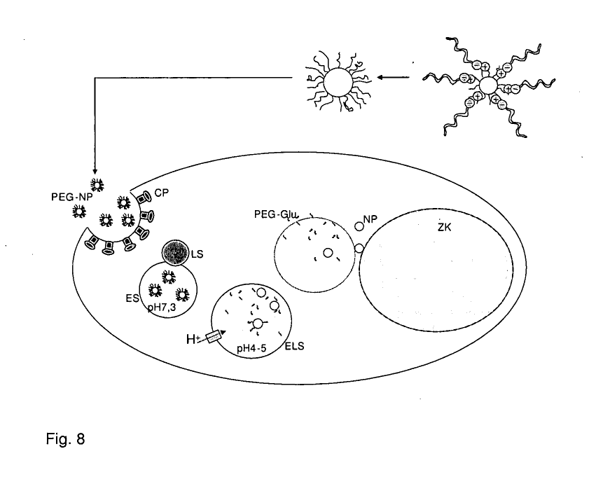

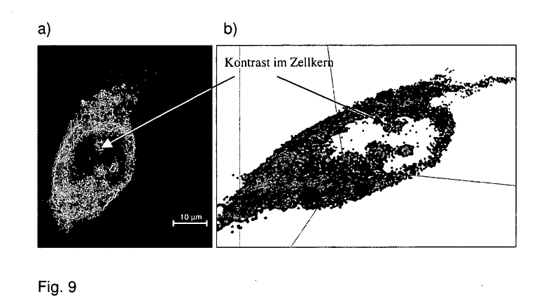

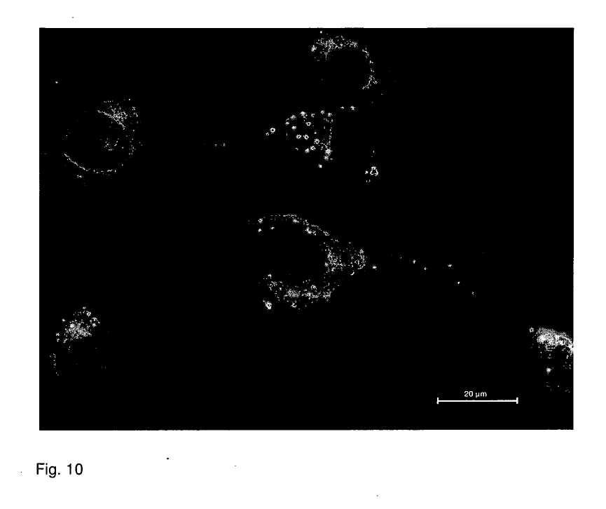

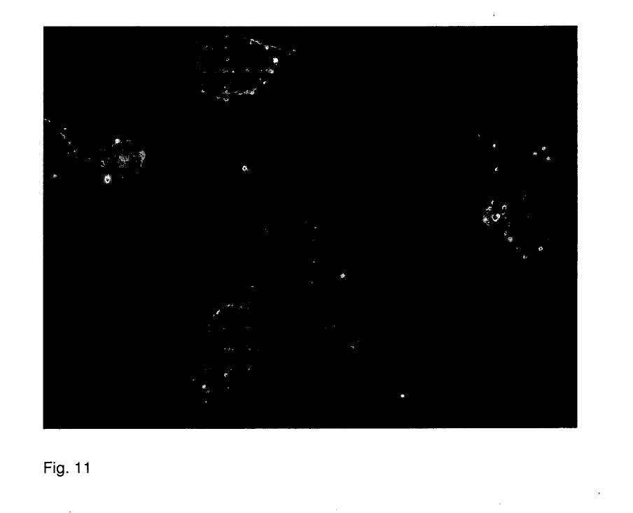

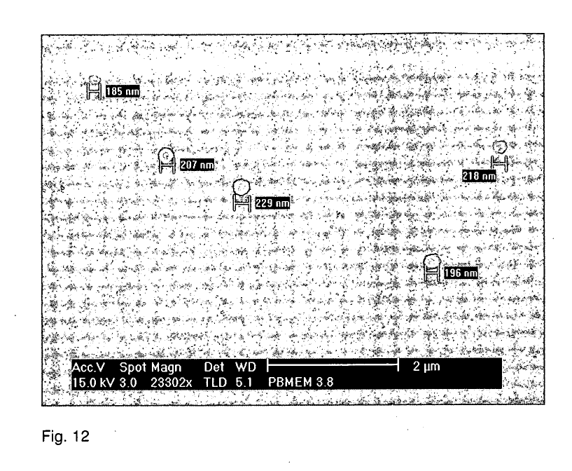

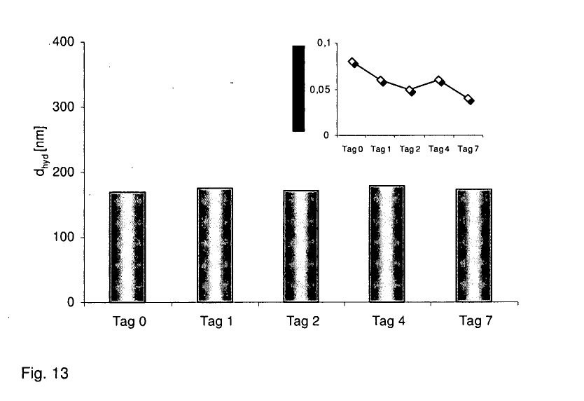

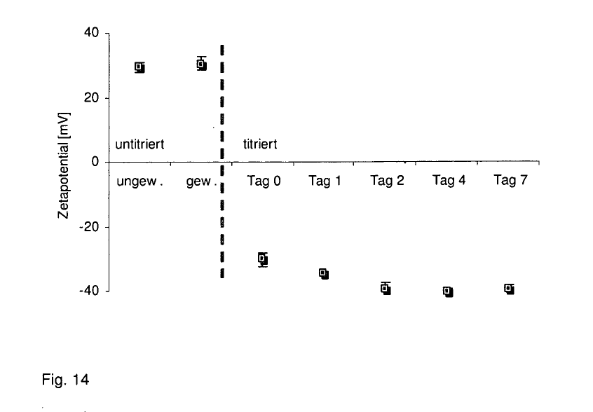

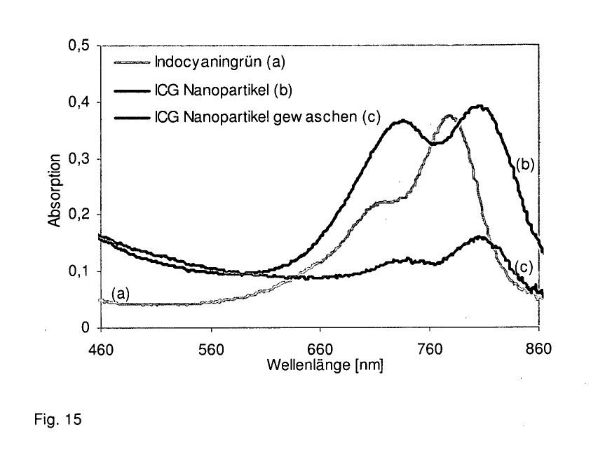

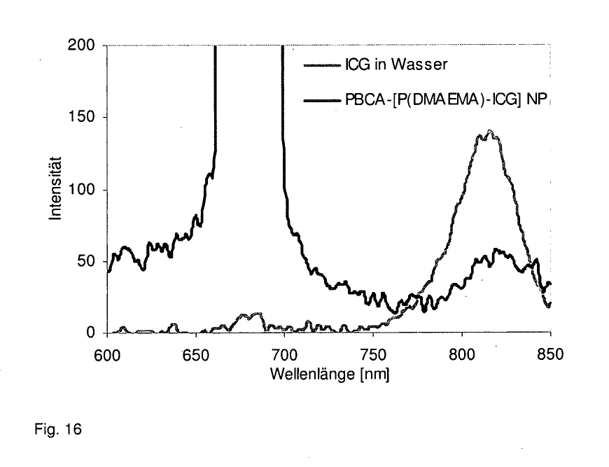

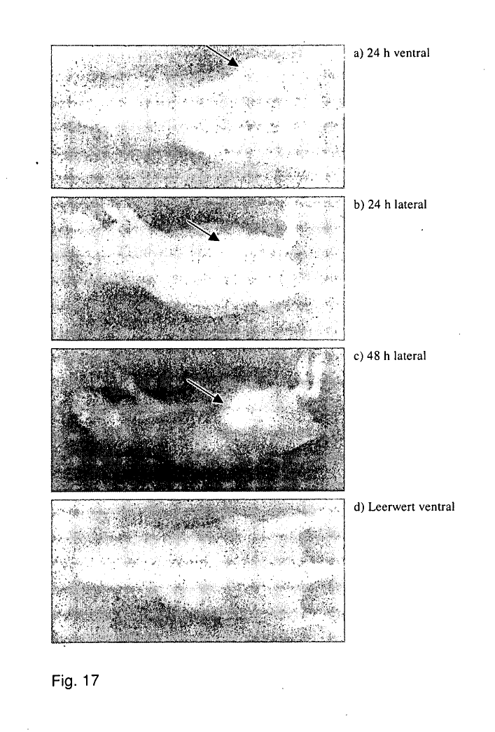

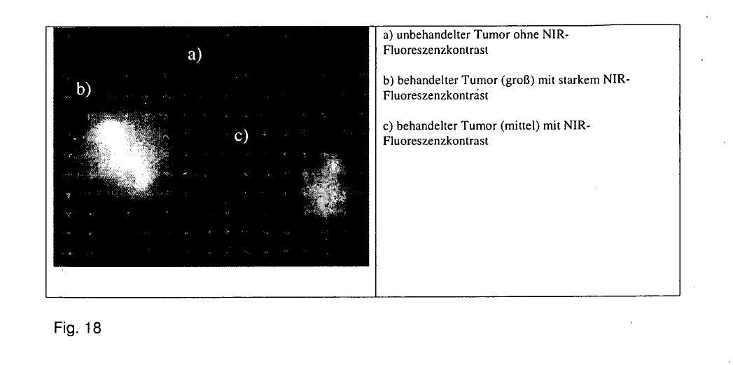

The figure shows NIR fluorescence contrasts a) of an untreated tumor without NIR fluorescence contrast, b) of a large, treated tumor and c) of a medium-size, treated tumor ex vivo 48 h after treatment.

1) A polymer nanoparticle with a cationic surface potential, containing a cationic polymer and a polymer that is sparingly water-soluble, characterized in that said polymer nanoparticle contains diagnostic and/or therapeutic agents.

2) The polymer nanoparticle as claimed in claim 1, characterized in that it comprises a precipitated aggregate.

3) The polymer nanoparticle as claimed in claim 1, characterized in that the sparingly water-soluble polymer is a polycyanoacrylate, polyalkylcyanoacrylate (PACA), polyester, alginic acid, hyaluronic acid, polysialic acid, acid cellulose derivatives, acid starch derivatives, polysaccharides, polymeric proteins, polyamides, polyanhydrides, polyorthoesters, polycaprolactones, polyphosphoric acid, poly(amide-enamines), azo polymers, polyurethanes, polyorthoesters, dendrimers, pseudopolyamino acids or all mixtures and copolymers of said compounds.

4) The polymer nanoparticle as claimed in claim 1, characterized in that the sparingly water-soluble polymer is a polybutylcyanoacrylate (PBCA).

5) The polymer nanoparticle as claimed in claim 1, containing a cationically modified polyacrylate P(DMAEMA), diethylaminoethyl-modified dextrans, hydroxymethylcellulose trimethylamine, polylysine, protamine sulfate, hydroxyethylcellulose trimethylamine, polyallylamines, protamine chloride, polyallylamine hydrated salts, polyamines, polyvinylbenzyl trimethylammonium salts, polydiallyldimethylammonium salts, polyimidazoline, polyvinylamine and polyvinylpyridine, polyethyleneimine (PEI), putrescine (butane-1,4-diamine), spermidine (N-(3- aminopropyl)butane-1,4-diamine), spermine (N,N′-bis(3- aminopropyl)butane-1,4-diamine), dimethylaminoethylacrylate, poly-N,N-dimethylaminoethylmethacrylate, dimethylaminopropylacrylamide, dimethylaminopropylmethacrylamide, dimethylaminostyrene, vinylpyridine and methyldiallylamine, poly-DADMAC, guar, or deacetylated chitin and the corresponding salts, which can form with suitable inorganic or low-molecular organic acids.

6) The polymer nanoparticle as claimed in claim 5, containing a cationically modified polyacrylate P(DMAEMA),

7) The polymer nanoparticle as claimed in claim 5, containing a polyethyleneimine.

8) The polymer nanoparticle as claimed in claim 1, wherein the surface is modified electrostatically.

9) The polymer nanoparticle as claimed in claim 1, wherein the surface is modified with Glu(10)-b-PEG(110).

10) The polymer nanoparticle as claimed in claim 1, wherein the diagnostic and/or therapeutic agent is negatively charged and is encapsulated as an ion pair with the cationic polymer in the particle.

11) The polymer nanoparticle as claimed in claim 1, wherein the diagnostic agent comprises a fluorescent dye.

12) The polymer nanoparticle as claimed in claim 1, wherein the diagnostic agent comprises a fluorescent NIR dye.

13) The polymer nanoparticle as claimed in claim 1, wherein the diagnostic agent comprises a carbocyanine dye.

14) The polymer nanoparticle as claimed in claim 1, wherein the diagnostic agent comprises TSC (tetrasulfocyanine).

15) The polymer nanoparticle as claimed in claim 1, wherein the diagnostic agent comprises IDCC (indodicarbocyanine).

16) The polymer nanoparticle as claimed in claim 1, wherein the diagnostic agent comprises ICG (Indocyanine Green).

17) The polymer nanoparticle as claimed in claim 1, wherein the therapeutic agent comprises a substance for the treatment of neoplastic diseases or diseases with inflammatory reactions.

18) The polymer nanoparticle as claimed in claim 1, which provides a target structure.

19) The polymer nanoparticle as claimed in claim 18, characterized in that the target structure possesses a negatively charged moiety and is applied to the cationic particle surface by electrostatic interactions.

20) The polymer nanoparticle as claimed in claim 18, wherein the target structure can comprise an antibody, a protein, a polypeptide, a polysaccharide, a DNA molecule, an RNA molecule, a chemical unit, a nucleic acid, a lipid, a carbohydrate or combinations of the aforementioned.

21) The polymer nanoparticle as claimed in claim 1, wherein the size of the particles is in the range 1-800 nm.

22) The polymer nanoparticle as claimed in claim 1, wherein the size of the particles is in the range 5-800 nm.

23) The polymer nanoparticle as claimed in claim 1, wherein the size of the particles is in the range 1-500 nm.

24) The polymer nanoparticle as claimed in claim 1, wherein the size of the particles is in the range 5-500 nm.

25) The polymer nanoparticle as claimed in claim 1, wherein the size of the particles is in the range 1-300 nm.

26) The polymer nanoparticle as claimed in claim 1, wherein the size of the particles is in the range 5-300 nm.

27) The polymer nanoparticle as claimed in claim 1, wherein the size of the particles is in the range 10-300 nm

28) A method of using the polymer nanoparticle as claimed in claim 1 comprising diagnosing or treating diseases, neoplastic diseases or diseases with inflammatory reactions with said polymer nanoparticle.

29) A method of production of the polymer nanoparticle as claimed in claim 1, characterized in that the following process steps are carried out:
Dissolution of the cationic polymer in an organic solvent or a solvent/water mixture
Dissolution of the water-insoluble polymer in an organic solvent
Dissolution of the active substance (diagnostic agent or therapeutic agent) in an organic solvent or a solvent/water mixture
Preparation of a completely dissolved mixture of cationic polymer, water-insoluble polymer and active substance
Adding the mixture to a surfactant-containing solution, with spontaneous formation of precipitated aggregates
Removal of the solvent
Electrostatic surface modification of the particles by adding together the nanoparticle dispersion and the modifying agent in suitable amounts (optional).

30) A method of using a nanoparticle as claimed in claim 1 which comprises producing a pharmaceutical preparation/pharmaceutical form using pharmaceutically acceptable excipients.

31) A method of claim 30, wherein the pharmaceutical preparation is administered via a suitable application system to humans or animals via a suitable route of administration.
